(19) United States
(12) Patent Application Publication
(10) Pub. No.: US 2006/0106117 A1
(43) Pub. Date: May 18, 2006

(54) Compound and method for prevention and/or treatment of vaginal infections

(75) Inventors: Shu Ping Yang, Alpharetta, GA (US); Lei Huang, Duluth, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/987,463

(22) Filed: Nov. 12, 2004

(51) Int. Cl. A61K 31/045

(52) U.S. Cl. 514/738

(57) ABSTRACT

The invention provides a method for treating and/or preventing vaginal infections, such as bacterial vaginosis, by administering xylitol to a subject in need of treatment. A composition containing xylitol for treating vaginal infections is also disclosed. It has been found that xylitol is capable of selectively inhibiting the growth of Gardnerella vaginalis, the main pathogen in bacterial vaginosis, while not inhibiting the growth of Lactobacillus acidophilus, the dominant bacteria in a healthy vaginal ecosystem. Xylitol, a natural five-carbon sugar polyol, is safe and cost-effective, and can be used alone or incorporated into different vaginal health products to treat and/or prevent bacterial vaginosis.

### BACKGROUND OF THE INVENTION

The vaginal ecosystem is a finely balanced environment maintained by a complex interaction among vaginal flora. A variety of bacteria, yeasts and other micro-organisms occur naturally in the vagina's environment. Lactobacillus acidophilus is the dominant bacteria in a healthy vaginal ecosystem, and it maintains an acidic environment of the vagina through the production of lactic acid. Lactic acid and hydrogen peroxide produced by Lactobacilli are toxic to anaerobic bacteria and other pathogenic bacteria in the vagina. The vaginal balance can be upset by external factors such as antibiotics, stress, illness and hormonal changes, and insults that decrease Lactobacilli result in an in overgrowth of pathogenic organisms in the vagina.

It is reported that 13 million women experience vaginal infections each year in the US. More than 75% of women will have at least one infection in their lives, and 50% of these women will have a recurrence of the infection (http://www.stopgettingsick.com/templates/news_template.cfm/1671).

Bacterial vaginosis, the most common vaginal infection, is caused by an overgrowth of a variety of bacterial species, particularly anaerobes. Gardnerella vaginalis is the main pathogen in bacterial vaginosis.

Bacterial vaginosis generally shows little or no inflammation of the vaginal epithelium and resembles more of an alteration of the bacterial vaginal environment than a real and proper infection of tissues or epithelium. This pathology is currently treated mainly with metronidazole, clindamycin or ampicillin administered orally, but this method of use by the systemic route is frequently accompanied by serious side effects. For example, metronidazole exhibits serious side effects, particularly on the blood and on the central nervous system, so much that in certain types of patients it has been necessary to discontinue the treatment and authorities in the medical field have recommended that women who use metronidazole should not breast feed (Martindale, The Extra Pharmacopoeia, 29th Edition, 1989, page 667).

Clindamycin also exhibits serious side effects, particularly on the gastrointestinal tract, with serious forms of diarrhea and pseudo-membranous colitis that can even lead to the death of the patient (Martindale, pages 198-199).

Antibiotic treatment may also kill beneficial bacteria such as lactobacilli, which maintains an acidic environment in the vagina through the production of lactic acid. This may result in a pH increase in the vaginal environment and the possibility of reoccurrence of bacterial vaginosis, or even increase the risk for other vaginal infections like the common yeast infection.

There is therefore a need for a suitable compound or composition that can treat and/or prevent vaginal infections without the side effects of known treatments.

### SUMMARY OF THE INVENTION

In response to the problems discussed above, it has been found that xylitol is capable of selectively inhibiting and/or killing pathogens such as Gardnerella vaginalis without affecting Lactobacilli growth. This compound is therefore suitable for use as an active ingredient in a method of treating and/or preventing vaginal infections, and in particular, bacterial vaginosis.

According to a first aspect of the invention, a method of treating a vaginal infection using xylitol is provided. The xylitol may be in the form of a solution, a powder and/or a crystal structure. The xylitol may be used alone or in a therapeutic amount in a composition, in the form of a foam, a cream, a gel, a moisturizer, a jelly, a spray, a suppository, a vaginal capsule, sponge, film, tablet or ovule or any other vaginal health product. The composition may also include a suitable diluent, excipient and/or auxiliary. The composition may also be applied to a feminine hygiene product such as tampons, feminine pads, feminine wipes, and vaginal inserts.

In general, the xylitol is present in the composition in an amount of from about 0.1 to about 20 weight percent, more preferably in an amount of from about 1 to about 10 weight percent, more preferably in an amount of from about 3 to about 7 weight percent and even more preferably in an amount of about 5 weight percent.

The method comprises the step of administering the composition topically to a subject in need thereof, so as to inhibit the growth of Gardnerella vaginalis without inhibiting the growth of Lactobacillus acidophilus.

According to a second aspect of the invention, a composition for treating a vaginal infection is described. The composition comprises a therapeutically effective amount of xylitol and is substantially as described above.

According to a third aspect of the invention, the use of xylitol in a method of manufacturing a medicament for treating and/or preventing a vaginal infection is described.

### BRIEF DESCRIPTION OF THE DRAWINGS

FIG. 1 shows the effects of xylitol on Gardnerella Vaginalis (OD) after 2, 4, 6, and 24 hours treatment (n=4, * represents P<0.05); and

FIG. 2 shows the effects of xylitol on Lactobacillus acidophilus (OD) after 2, 4, 6, and 24 hours treatment (n=4).

### DETAILED DESCRIPTION OF THE INVENTION

The invention provides a method for treating a vaginal infection by topically administering a therapeutic amount of xylitol to a subject in need thereof. Xylitol is shown below to selectively inhibit and/or kill pathogens like Gardnerella vaginalis, without affecting Lactobacilli growth, the presence of the former being a cause of bacterial vaginosis and the latter being a desirable presence in the ecosystem of the vagina.

Xylitol is a five-carbon sugar polyol, small amounts of which occur naturally in plums, strawberries and raspberries. It has also been called “birch sugar”, as it can be produced from xylan derived from birch wood chips. It is equal in sweetness to sucrose, with 1 g yielding 4.06 kcal.

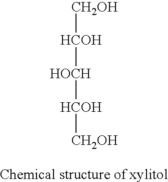

Xylitol has been known since the late Ninteenth Century. German and French researchers were the first to produce xylitol about 100 years ago, when a syrup-like mixture was made. However, xylitol was not manufactured in a crystalline form until World War Two, and its status remained that of a research compound until it was used as an alternate sweetener during World War Two, due to war-associated sugar shortages.

The discovery of xylitol's insulin-independent properties resulted in it being introduced into diabetic diets, which was its primary use up until about 1975, when xylitol was first used as a sugar-free chewing gum. Since then, xylitol's other biological properties have been continually explored. There is increasing global awareness of xylitol's significant dental benefits (http://herkules.oulu.fi//isbn9514267796. Terhi Tapiainen (2002) Microbiological Effects And Clinical Use Of Xylitol In Preventing Acute Otitis Media), and the compound is widely used as a sweetener in sugar-free candy, gums and mints. Xylitol is believed to be a safe compound, and high levels of it are to be found in dental products such as toothpaste and chewing gums.

Xylitol is a normal intermediate of human metabolism, and several grams of it are produced daily by the liver (Terhi Tapiainen; Ylikahri R (1979) Metabolic and nutritional aspects of xylitol. Adv Food Res 25:159-80). Exogenous xylitol is metabolized to glucose and glucogen or pyruvate and lactate in the liver. Many bacteria are nevertheless unable to utilize xylitol as an energy source, and its presence is harmful to some bacteria despite the availability of an alternative energy source such as glucose (Assev S, Vegarud G, Rölla G (1980) Growth inhibition of Streptococcus mutans strain OMZ 176 by xylitol. Acta Path Microbiol Scand 88:61-63; Knuuttila M L, Mäkinen K (1975) Effect of xylitol on the growth and metabolism of Streptococcus mutans. Caries Res 9:177-89). More recently, xylitol has also been described for use in reducing ionic strength and activating endogenous antimicrobials to treat cystic fibrosis (U.S. Pat. No. 6,716,819 to Welsh and Zabner), mucosal yeast infections (U.S. Pat. No. 6,414,035 to Munita et al.) and respiratory infections (U.S. Pat. No. 6,066,677 to Uhari and Kontiokari).

It has been shown in several studies that xylitol significantly reduces the growth of Streptococcus mutans in the presence of glucose or sucrose (Assev S et al. (1980); Knuuttila M L et al.; Edwardsson S, Birkhed D, Mejare B (1977) Acid production from Lycasin, maltitol, sorbitol and xylitol by oral streptococci and lactobacilli. Acta Odontol Scand 35:257-263; Assev S, Waler S M, Rölla G (1983) Further studies on the growth inhibition of some oral bacteria by xylitol.

Acta Path Immunol Scand 91:261-265; Vadeboncoeur C, Trahan L, Mouton C, Mayrand D (1983) Effect of xylitol on the growth and glycolysis of acidogenic oral bacteria. J Dent Res 62:882-884). Xylitol has also been shown to reduce the growth of Streptococcus salivarius, Streptococcus sanguis and some strains of Escherichia coli, Saccharomyces cerevisae and Salmonella typhii (London J, Hausman S (1982) Xylitol-mediated transient inhibition of ribitol utilization by Lactobacillus casei. J Bacteriol 150:657-661; Reiner A M (1977) Xylitol and D-arabitol toxicities due to derepressed fructose, galactitol, and sorbitol phosphotransferases of Escherichia coli. J Bacteriol; 132:166-173; Mäkinen K, Söderling E (1981) Effect of xylitol on some food-spoilage micro-organisms. J Food Sci 46:950-951; Macfadyen L P, Dorocicz I R, Reizer J, Saier M H, Jr., Redfield R J (1996) Regulation of competence development and sugar utilization in Haemophilus influenzae Rd by a phosphoenolpyruvate:fructose phosphotransferase system. Mol Microbiol 21:941-952).

The microbiological mechanism of the action of xylitol has not been fully discovered yet. The most detailed study found in the literature relates only to Streptococcus mutans. This study showed that xylitol can be transported into S. mutans where it is phosphorylated through a constitutive fructose phosphotransferase system. The phosphotransferase system in bacteria regulates many metabolic processes and the expression of various genes (Saier M H, Jr., Reizer J (1994) The bacterial phosphotransferase system: new frontiers 30 years later. Mol Microbiol 13:755-764). It is thus likely that xylitol can retard or inhibit bacteria growth through disturbing the metabolic processes in viable bacteria. It was also found that even very low concentrations of xylitol can damage the ultrastructure of viable S. mutans bacteria (Tuompo H, Meurman J H, Lounatmaa K, Linkola J (1983) Effect of xylitol and other carbon sources on the cell wall of Streptococcus mutans. Scand J Dent Res; 91:17-25); and their protein synthesis is also disturbed, which implies that xylitol acts as a strong metabolic inhibitor for this species. Xylitol also affect polysaccharide synthesis in S. mutans, resulting in decreased bacterial adherence (Söderling E, Alaräisänen L, Scheinin A, Mäkinen KK (1987) Effect of xylitol and sorbitol on polysaccharide production by and adhesive properties of Streptococcus mutans. Caries Res 21:109-116). Since bacteria adhere to host cells through carbohydrate-binding proteins (Ofek I, Sharon N (1990) Adhesins as lectins: specificity and role in infection. Curr Top Microbiol Immunol 151:91-11), extracellular xylitol may disturb the binding process by acting as a receptor analogue for the host cell, which could result in decreased adherence (Soderling et al. (1987); Kontiokari T, Uhari M, Koskela M (1998) Antiadhesive effects of xylitol on otopathogenic bacteria. J Antimicrob Chemother 41:563-565).

The applicants have found that xylitol has the effect of selectively inhibiting Gardnerella vaginalis while not inhibiting Lactobacillus acidophilus, and is therefore a suitable for compound for treating and/or preventing vaginal infections, and in particular bacterial vaginosis.

The xylitol can be used in the form of a solution, powder and/or crystal structure, either alone or as part of a composition. It is typically administered topically as part of a composition which is in the form of a foam, cream, gel, moisturizer, spray, vaginal capsule, vaginal ovule or any other vaginal health product. The composition optionally also includes suitable diluents, excipients and/or auxiliaries, which are well known in the art.

It is believed that a xylitol containing polymer could similarly be used with the same or similar activity against Gardnerella vaginalis while not inhibiting Lactobacillus acidophilus in the same concentrations. This xylitol containing polymer could be a polyxylitol or a mixed polysaccharide with at least 50% xylitol units. Alternatively, the xylitol containing polymer could contain mainly a carrier polymer such as, but not limited to, polyethyleneglycol (PEG) or a starch polysaccharide, with xylitol end groups. Lastly, the xylitol containing polymer could contain a backbone carrier polymer as described previously, with multiple xylitol pendant groups along the chain. Other active constituents have previously been attached to a polymers and maintained similar activity while providing other benefits in terms of ease of formulation, speed of diffusion and/or other benefits.

The composition can be applied to a vaginal insert, tampon, wipe or pad.

In general, the xylitol is present in the composition in an amount of from about 0.1% to about 10 weight percent, more preferably in an amount of from about 1% to about 7.5 weight percent, and even more preferably in an amount of about 5 weight percent.

The present invention is further described by the following examples. Such examples, however, are not to be construed as limiting in any way, either the spirit or scope of the invention.

### EXAMPLES

Microorganisms and Culture Media:

A sample of Gardnerella vaginalis, the pathogenic bacterium found in bacterial vaginosis, was obtained from the American Type Culture Collection (ATCC), catalog number 14018. The culture medium was ATCC medium 70 and Casman's medium (BD 229010) with 5% rabbit blood.

A sample of Lactobacillus acidophilus, a desirable bacterium in the vaginal ecosystem, was also obtained from the American Type Culture Collection (ATCC), catalog number 4354, and was cultured in ATCC medium 416.

### Example 1

### Effect of Xylitol on the Growth of Gardnerella Vaginalis and Lactobacillus Acidoohilus—zone-of-inhibition test

A microorganism culture of 10⁵ cfu (colony forming units)/ml in a 1× phosphate buffered saline (PBS) solution (diluted from 10× PBS LIQUID CONCENTRATE from VWR Cat# EM-6507) was used. One milliliter of the solution was plated on proper agar plates, depending on which microorganism was being tested. The agar plates were incubated at 35° C for four hours. Three 4 millimeter diameter wells were then punched in each agar plate. A test sample of 100 microliters in sterilized 2-N-morpholino ethane sulfonic (MES, pH=4.7) buffer (0.1 M 2-[morpholino]-ethanesulfonic acid, 0.9% NaCl, pH 4.7, prepared from BupH™ MES Buffer Saline Pack from Cat # 28390, Pierce Biotechnology, Inc., Rockford, Ill.) was added to one well of each plate. Into each of the other two wells were added MES buffer and 1% Benzyl Quats (diluted from BARDAC® 205M, from Lonza Inc., Fair Lawn, N.J.) as negative and positive control, respectively. The plates were incubated overnight at 35° C. The presence of a zone of microorganism inhibition was measured the following day for Gardnerella vaginalis and Lactobacillus acidophilus activity.

As shown in Table 1, xylitol at a concentration of 5% selectively inhibited Gardnerella vaginalis, while it did not affect the growth of Lactobacillus acidophilus. The positive control, 1% Benzyl Quats, inhibited both microorganisms, while MES buffer itself had no effect on either of the two microorganisms.

TABLE 1
Effect of xylitol on Gardnerella vaginalis and Lactobacillus acidophilus
with zone inhibition test, n = 2.

| Tested Compounds/ | | |
|---|---|---|
| Polymers | Gardnerella vaginalis | Lactobacillus acidophilus |
| 5% xylitol | 4 mm | 0 mm |
| 1% Benzyl Quats | 5 mm | 15 mm |
| MES buffer | 0 mm | 0 mm |

### Example 2

### Effect of Xylitol on the Growth of Gardnerella Vaginalis and Lactobacillus Acidophilus—Inhibition Tests in Solution by Measuring Optical Density

Test compounds were dissolved in culture media to form a suspension. Control or xylitol solutions (0.9 milliliters) were filtered and added into culture tubes, and to this was added 0.1 milliliter of either the Gardnerella vaginalis or Lactobacillus acidophilus suspension at a concentration of around 10⁶ cfu/milliliter. The culture tubes were then incubated overnight at 37° C., after which the optical density was measured at 2, 4, 6 and 24 hours at 590 nanometers by pippeting 100 microliters of the control or sample solutions into 96-well microplates, and then using a Molecular Devices of Sunnyvale, Calif. ThermoMax Microplate Reader to obtain the optical density readings at 340 nm or 590 nm wavelengths.

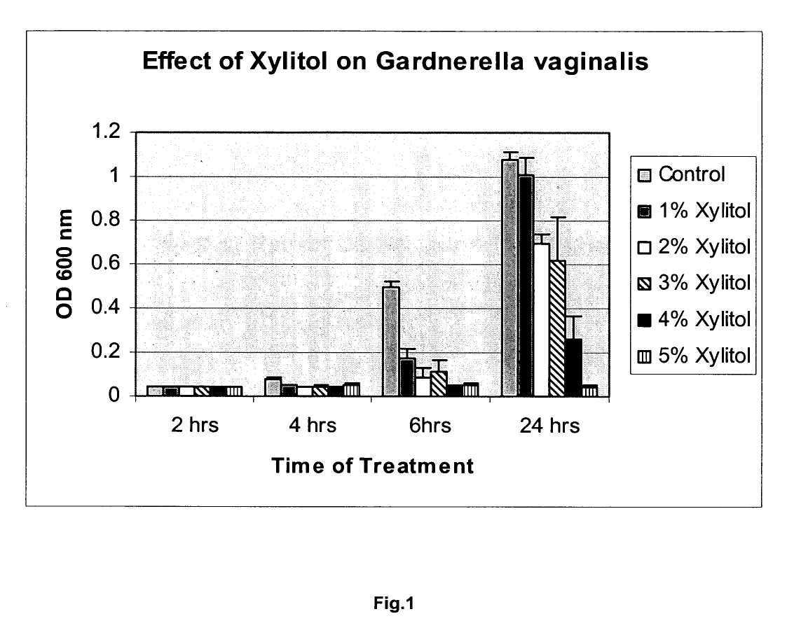

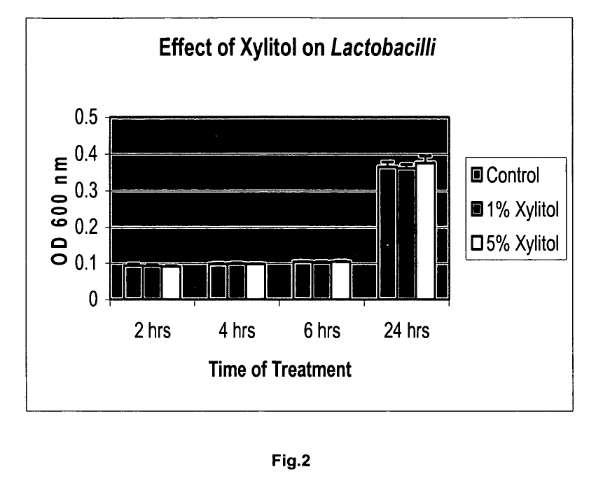

FIGS. 1 and 2 show the parallel study result that various concentrations of xylitol inhibiteding/killed Gardnerella vaginalis and Lactobacillus acidophilus at 2, 4, 6 and 24 hours by measuring optical density. Xylitol showed significant inhibition on the growth of Gardnerella vaginalis, which was inhibited as early as 2 hours after xylitol treatment. The inhibition effect remained evident throughout the 24 hour experimental period.

In contrast to the profound inhibition on the growth of Gardnerella vaginalis, xylitol did not show any significant inhibition on the growth of Lactobacillus acidophilus at any time (FIG. 2).

These optical density results were consistent with the plate counting data (example 3) and the zone-of-inhibition results (example 1).

In FIG. 1 the Y-axis is the measured optical density at a wavelength of 600 nm on a scale of 0 to 1.5 with divisions at each 0.5. The X-axis gives the results at each time period; 2, 4, 6 and 24 hours. At each time, the vertical bars represent, respectively from left to right, the control and 1, 2, 3, 4 and 5 percent xylitol.

In FIG. 2 the Y-axis is the measured optical density at a wavelength of 600 nm on a scale of 0. to 0.5 with divisions at each 0.1. The X-axis gives the results at each time period; 2, 4, 6 and 24 hours. At each time, the vertical bars represent, respectively from left to right, the control, 1 and 5 percent xylitol.

### Example 3

### Effect of Xylitol on the Growth of Gardnerella Vaginalis and Lactobacillus Acidophilus—Inhibition Tests in Solution by Plate Count

The test compounds were dissolved in culture media to form a suspension. Control or xylitol solutions (0.9 milliliters) were filtered and added into culture tubes; and to this was then added 0.1 milliliter of the Gardnerella vaginalis or Lactobacillus acidophilus suspension at a concentration of around 10⁶ cfu/milliliter. The culture tubes were incubated at 37° C. for 6 hours.

The samples in the culture tubes were then diluted at 1, 10 and 100 times, and 100 microliters of each dilution was plated onto agar plates with WASP (Whitely Automatic Spiral Plate) spiral plating equipment from Don Whitely Scientific Limited, USA. The plates were incubated overnight at 35° C., and the numbers of colonies were counted on each plate by either ProtoCol® from Synbiosis, Frederick, Md., USA Whitely Scientific Limited, USA or by hand count.

In this example, xylitol at concentrations of 1%, 2%, 3%, 4% and 5% was tested on its effect on Gardnerella vaginalis growth. After 6 hours of treatment, all five concentrations of the xylitol showed significant inhibition of Gardnerella vaginalis growth compared to a control group (Table 2). Over 99% inhibition was observed with the lowest concentration (1%), and the degree of inhibition enhanced with increased concentrations of xylitol.

TABLE 2
Effect of xylitol on Gardnerella Vaginalis after 6 hours treatment, n = 4

| Negative Control | 1% Xylitol | 2% Xylitol | 3% Xylitol | 4% Xylitol | 5% Xylitol |
|---|---|---|---|---|---|
| 2.32E+07 | *1.24E+05 | *1.64E+03 | *4.63E+02 | *1.03E+02 | *1.98E+01 |

*represents p < 0.05 compared to negative control group.

In contrast to the effect on Gardnerella vaginalis, xylitol did not show any significant inhibition on the growth of Lactobacillus acidophilus (Table 3). This data confirmed the result of zone inhibition test that xylitol selectively inhibits Gardnerella vaginalis without affecting Lactobacillus acidophilus.

TABLE 3
Effect of xylitol on Lactobacilli after 6 hours treatment, n = 4.

| Negative Control | 1% Xylitol | 5% Xylitol |
|---|---|---|
| 3.85 ± 0.44E+05 | 3.77 ± 0.49E+05 | 3.97 ± 0.36E+05 |

### Example 4

### Comparison of Xylitol with Other Polyol and Sugar Compounds

In order to examine whether other sugar compounds also have an inhibitory effect on Gardnerella Vaginalis, sorbitol and glucose were tested as described above. Table 4 shows parallel zone-of-inhibition results of the effects of sorbitol, glucose and xylitol on the growth of Gardnerella Vaginalis and Lactobacillus acidophilus. Neither sorbitol nor glucose showed any inhibition on the growth of Gardnerella vaginalis or Lactobacillus acidophilus, while xylitol once again showed an inhibitory effect on the growth of Gardnerella vaginalis but not on Lactobacillus acidophilus. The results suggest that xylitol's bacterial inhibition capability is unique among polyols and sugars.

TABLE 4
Effect of xylitol on Gardnerella vaginalis and Lactobacillus acidophilus
using a zone-of-inhibition test

| Tested Compounds/ | | |
|---|---|---|
| Polymers | Gardnerella vaginalis | Lactobacillus acidophilus |
| 10% xylitol | 4 mm | 0 mm |
| 10% sorbitol | 0 mm | 0 mm |
| 10% glucose | 0 mm | 0 mm |
| 1% Benzyl Quats | 5 mm | 15 mm |
| MES buffer | 0 mm | 0 mm |

The results of the above tests repeatedly show that different concentrations of xylitol are able to effectively inhibit Gardnerella vaginalis growth, without affecting the growth of Lactobacillus acidophilus. As xylitol is a naturally occurring, safe compound which is also cost-effective, it is ideally suited to be formulated into vaginal health products, such as tampons, pads, wipes, vaginal moisturizers, sprays, gels and so forth for preventing and/or treating bacterial vaginosis.

While the invention has been described in detail with respect to specific embodiments thereof, it will be appreciated by those skilled in the art that various alterations, modifications and other changes may be made to the invention without departing from the spirit and scope of the present invention. It is therefore intended that the claims cover or encompass all such modifications, alterations and/or changes.

What is claimed is:

1. A method of treatment of a vaginal infection which comprises administering topically to a subject in need of prevention or treatment a therapeutically effective amount of xylitol.

2. The method of claim 1, wherein the vaginal infection is bacterial vaginosis.

3. The method according to claim 1, wherein the xylitol is part of a composition selected from the group consisting of a foam, a cream, a gel, a jelly, a moisturizer, a spray, a suppository, a vaginal capsule, a vaginal tablet, a vaginal film, a vaginal sponge, or a vaginal ovule.

4. The method according to claim 1, wherein the xylitol is applied to a feminine hygiene product selected from the group consisting of tampons, feminine pads, feminine wipes, and vaginal inserts.

5. The method according to claim 1, wherein the xylitol is present in the composition in an amount of from about 0.1 to about 20 weight percent.

6. The method according to claim 1, wherein the xylitol is present in the composition in an amount of from about 1 to about 7.5 weight percent.

7. The method according to claim 1, wherein the xylitol is present in the composition in an amount of about 5 weight percent.

8. A composition for preventing or treating a vaginal infection, which comprises a therapeutically effective amount of xylitol.

9. The composition of claim 8, wherein the vaginal infection is bacterial vaginosis.

10. The composition of claim 8, wherein the xylitol is in a form selected from the group consisting of a solution, a powder and a crystal structure.

11. The composition of claim 8, which is in the form of a foam, a cream, a gel, a jelly, a moisturizer, a spray, a suppository, a vaginal capsule, a vaginal tablet, a vaginal film, a vaginal sponge, a vaginal ovule.

12. The composition of claim 8, which is applied to a tampon, pad, wipe or vaginal insert.

13. The composition of claim 8, wherein the xylitol is present in the composition in an amount of from about 0.1 to about 20 weight percent.

14. The composition of claim 8, wherein the xylitol is present in the composition in an amount of from about 1 to about 7.5 weight percent.

15. The composition of claim 8, wherein the xylitol is present in the composition in an amount of about 5 weight percent.

16. The use of xylitol in a method of making a medicament for use in a method of preventing or treating a vaginal infection comprising administering topically a therapeutically effective amount of the xylitol to a patient in need thereof.

17. The use of xylitol according to claim 16, wherein the vaginal infection is bacterial vaginosis.

18. The use of xylitol according to claim 16, wherein the xylitol is in a form selected from the group consisting of a solution, a powder and a crystal structure.

19. The use of xylitol according to claim 16, wherein the medicament is in the form of of a foam, a cream, a gel, a jelly, a moisturizer, a spray, a suppository, a vaginal capsule, a vaginal tablet, a vaginal film, a vaginal sponge, a vaginal ovule.

20. The use of xylitol according to claim 16, wherein the medicament is applied to a tampon, pad, wipe or vaginal insert.
